(19) United States
(12) Patent Application Publication
(10) Pub. No.: US 2022/0406432 A1
(43) Pub. Date: Dec. 22, 2022

(54) WALKING TRAINING SYSTEM, CONTROL METHOD THEREOF, AND CONTROL PROGRAM

(71) Applicant: TOYOTA JIDOSHA KABUSHIKI KAISHA, Toyota-shi (JP)

(72) Inventors: Mitsugu KIKUCHI, Miyoshi-shi (JP); Taiga MATSUMOTO, Toyota-shi (JP)

(21) Appl. No.: 17/658,038

(22) Filed: Apr. 5, 2022

(30) Foreign Application Priority Data
Jun. 16, 2021 (JP) 2021-100055

(51) Int. Cl. G16H 20/30; A63B 22/02; A61H 1/02; A61B 5/103; G16H 40/63

(52) U.S. Cl. CPC G16H 20/30; A63B 22/02; A61H 1/0262; A61B 5/1038; G16H 40/63; A63B 2220/05; A61B 2562/0252; A61B 2505/09; A63B 2220/833; A61B 5/0077

(57) ABSTRACT

A walking training system according to the present embodiment includes: a treadmill; a load distribution sensor; an imaging device that takes an image of a trainee; a specifying unit that specifies, from the image taken by the imaging device, whether a load detected by a load distribution sensor is a load received from a sole of a right leg of the trainee or a load received from a sole of a left leg of the trainee; and a determination unit that determines, based on a status of a load received from a sole of one leg, out of the right leg and the left leg of the trainee during walking training, in a standing state that is detected by the load distribution sensor, whether the sole of the one leg in the standing state is located within a load detection area of the load distribution sensor.

### CROSS-REFERENCE TO RELATED APPLICATION

This application claims priority to Japanese Patent Application No. 2021-100055 filed on Jun. 16, 2021, incorporated herein by reference in its entirety.

### BACKGROUND

### 1. Technical Field

The present disclosure relates to a walking training system, a control method thereof, and a control program.

### 2. Description of Related Art

Japanese Patent No. 6187208 (JP 6187208 B) discloses a walking rehabilitation system including a treadmill, a floor reaction force sensor for measuring a reaction force applied to the treadmill, a leg robot attached to a lower leg of a user, a distance image camera that captures a distance of the lower leg to which the leg robot is attached, and a load estimation unit that estimates sole loads of the right and left lower legs of the user based on the measured value of the floor reaction force sensor and the image captured by the distance image camera.

### SUMMARY

In the related art, it is not determined whether the user (trainee) is normally walking within a load detection area of the floor reaction force sensor. Therefore, for example, even when the user walks outside the load detection area of the floor reaction force sensor, the sole loads of the right and left lower legs of the user are estimated without considering that fact. Accordingly, in the related art, there is a problem that the reliability of the estimation result of the load received from the sole of the trainee is lowered. As a result, in the related art, there is a possibility that the walking state of the trainee cannot be accurately estimated, for example, and effective walking training cannot be provided to the trainee.

The present disclosure has been made in view of the above background, and it is an object thereof to provide a walking training system capable of improving the reliability of the detection result of the load received from the sole of the trainee by determining whether the trainee is normally walking within the load detection area of the load distribution sensor, a control method thereof, and a control program.

A walking training system according to an embodiment of the present disclosure includes: a treadmill; a load distribution sensor that is provided on a lower side of a belt of the treadmill so as not to move together with the belt and that detects a distribution of a load received from a sole of a trainee riding on the belt of the treadmill; an imaging device that takes an image of the trainee; a specifying unit that specifies, from the image taken by the imaging device, whether the load detected by the load distribution sensor is a load received from a sole of a right leg of the trainee or a load received from a sole of a left leg of the trainee; and a determination unit that determines, based on a status of a load received from a sole of one leg, out of the right leg and the left leg of the trainee during walking training, in a standing state that is detected by the load distribution sensor, whether the sole of the one leg in the standing state is located within a load detection area of the load distribution sensor. The walking training system can determine whether the trainee is normally walking within the load detection area of the load distribution sensor. Therefore, for example, the walking training system can exclude the load received from the sole of the leg of the trainee that is determined as walking outside the load detection area of the load distribution sensor, from the reference that is used when estimating the walking state of the trainee. That is, the walking training system can improve the reliability of the detection result of the load received from the sole of the trainee. As a result, the walking training system can accurately estimate the walking state of the trainee, for example, and thus can provide effective walking training to the trainee.

When the load received from the sole of the one leg, out of the right leg and the left leg of the trainee during walking training, in the standing state that is detected by the load distribution sensor is smaller than a predetermined load, the determination unit may determine that the sole of the one leg in the standing state is located outside the load detection area of the load distribution sensor.

When a distribution area of the load received from the sole of the one leg, out of the right leg and the left leg of the trainee during walking training, in the standing state that is detected by the load distribution sensor is smaller than a predetermined area, the determination unit may determine that the sole of the one leg in the standing state is located outside the load detection area of the load distribution sensor.

When the load received from the sole of the one leg, out of the right leg and the left leg of the trainee during walking training, in the standing state that is detected by the load distribution sensor is detected in an end area that is set along an outer periphery of the load detection area of the load distribution sensor, the determination unit may determine that the sole of the one leg in the standing state is located outside the load detection area of the load distribution sensor.

The walking training system may further include an estimation unit that estimates a walking state of the trainee based on a load received from a sole of each of the right leg and the left leg of the trainee that is detected by the load distribution sensor and specified by the specifying unit.

When the determination unit determines that the sole of the one leg is located outside the load detection area of the load distribution sensor, the estimation unit may estimate a walking state of the one leg based on a change in a load received from a sole of another leg that is detected by the load distribution sensor.

When the determination unit determines that the sole of the one leg is located outside the load detection area of the load distribution sensor, the estimation unit may estimate a walking state of the one leg based on information on a past load change of the one leg.

The walking training system may further include: a robot leg attached to at least one leg of the trainee; and a control unit that controls extension of the robot leg based on an estimation result by the estimation unit.

A method for controlling a walking training system according to an embodiment of the present disclosure includes: a step of using a load distribution sensor that is provided on a lower side of a belt of a treadmill so as not to move together with the belt, to detect a distribution of a load received from a sole of a trainee riding on the belt of the treadmill; a step of taking an image of the trainee using an imaging device; a step of specifying, from the image taken by the imaging device, whether the load detected by the load distribution sensor is a load received from a sole of a right leg of the trainee or a load received from a sole of a left leg of the trainee; and a step of determining, based on a status of a load received from a sole of one leg, out of the right leg and the left leg of the trainee during walking training, in a standing state that is detected by the load distribution sensor, whether the sole of the one leg in the standing state is located within a load detection area of the load distribution sensor. With the method for controlling a walking training system, it is possible to determine whether the trainee is normally walking within the load detection area of the load distribution sensor. Therefore, for example, it is possible to exclude the load received from the sole of the leg of the trainee that is determined as walking outside the load detection area of the load distribution sensor, from the reference that is used when estimating the walking state of the trainee. That is, the method for controlling a walking training system can improve the reliability of the detection result of the load received from the sole of the trainee. As a result, the method for controlling a walking training system can accurately estimate the walking state of the trainee, for example, and thus can provide effective walking training to the trainee.

A control program according to an embodiment of the present disclosure causes a computer to execute: a process of using a load distribution sensor that is provided on a lower side of a belt of a treadmill so as not to move together with the belt, to detect a distribution of a load received from a sole of a trainee riding on the belt of the treadmill; a process of taking an image of the trainee using an imaging device; a process of specifying, from the image taken by the imaging device, whether the load detected by the load distribution sensor is a load received from a sole of a right leg of the trainee or a load received from a sole of a left leg of the trainee; and a process of determining, based on a status of a load received from a sole of one leg, out of the right leg and the left leg of the trainee during walking training, in a standing state that is detected by the load distribution sensor, whether the sole of the one leg in the standing state is located within a load detection area of the load distribution sensor. The control program can determine whether the trainee is normally walking within the load detection area of the load distribution sensor. Therefore, for example, the control program can exclude the load received from the sole of the leg of the trainee that is determined as walking outside the load detection area of the load distribution sensor, from the reference that is used when estimating the walking state of the trainee. That is, the control program can improve the reliability of the detection result of the load received from the sole of the trainee. As a result, the control program can accurately estimate the walking state of the trainee, for example, and thus can provide effective walking training to the trainee.

The present disclosure can provide a walking training system capable of improving the reliability of the detection result of the load received from the sole of the trainee by determining whether the trainee is normally walking within the load detection area of the load distribution sensor, a control method thereof, and a control program.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is an overall conceptual diagram showing a configuration example of a walking training device according to a first embodiment;

FIG. 2 is a schematic side view of a part of a treadmill provided in the walking training device shown in FIG. 1;

FIG. 3 is a schematic perspective view showing a configuration example of a walking assist device provided in the walking training device shown in FIG. 1;

FIG. 4 is a block diagram showing a system configuration example of the walking training device shown in FIG. 1;

FIG. 5 is a diagram illustrating a problem of a method for estimating a walking state of a trainee according to the related art;

FIG. 6 is a diagram illustrating the problem of the method for estimating the walking state of the trainee according to the related art;

FIG. 7 is a diagram illustrating a first example of a method for determining whether a sole of a leg of the trainee in a standing state is located within a load detection area of a load distribution sensor by the walking training device shown in FIG. 1;

FIG. 8 is a diagram illustrating the first example of the method for determining whether the sole of the leg of the trainee in the standing state is located within the load detection area of the load distribution sensor by the walking training device shown in FIG. 1;

FIG. 9 is a timing chart illustrating a second example of the method for determining whether the sole of the leg of the trainee in the standing state is located within the load detection area of the load distribution sensor by the walking training device shown in FIG. 1;

FIG. 10 is a timing chart illustrating a third example of the method for determining whether the sole of the leg of the trainee in the standing state is located within the load detection area of the load distribution sensor by the walking training device shown in FIG. 1;

FIG. 11 is a diagram illustrating the third example of the method for determining whether the sole of the leg of the trainee in the standing state is located within the load detection area of the load distribution sensor by the walking training device shown in FIG. 1;

FIG. 12 is a timing chart illustrating a first example of a method for estimating the walking state of the trainee by the walking training device shown in FIG. 1;

FIG. 13 is a timing chart illustrating a second example of the method for estimating the walking state of the trainee by the walking training device shown in FIG. 1; and

FIG. 14 is a timing chart illustrating a third example of the method for estimating the walking state of the trainee by the walking training device shown in FIG. 1.

### DETAILED DESCRIPTION OF EMBODIMENTS

Hereinafter, the present disclosure will be described through embodiments of the disclosure, but the disclosure according to the scope of the claims is not limited to the following embodiments. Moreover, not all of the configurations described in the embodiments are indispensable as means for solving the problem. For the sake of clarity, omission and simplification are made as appropriate in the following description and drawings. In the drawings, the same elements are designated by the same reference signs, and duplicate descriptions are omitted as necessary.

### First Embodiment

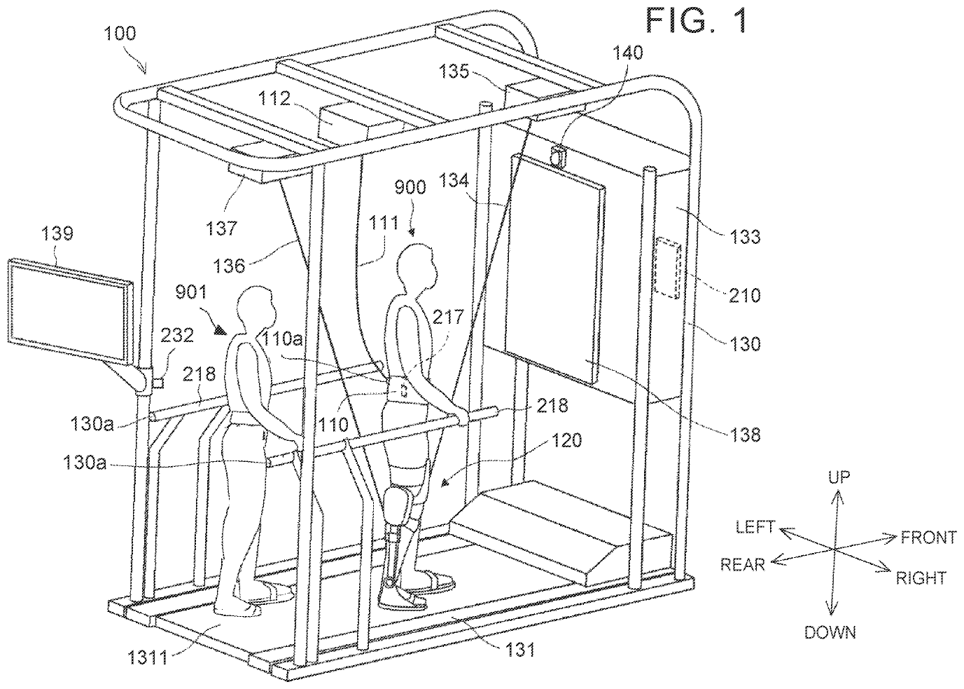

FIG. 1 is an overall conceptual diagram showing a configuration example of a walking training device according to a first embodiment. A walking training device 100 according to the present embodiment is a specific example of a rehabilitation support device that supports the rehab (rehabilitation) of a trainee (user) 900, and is particularly a specific example of a walking training device that supports walking training. The walking training device 100 is a device for the trainee 900 who is a hemiplegic patient suffering from paralysis in one leg to perform walking training in accordance with the guidance of a training staff 901. Here, the training staff 901 can be, for example, a therapist (physiotherapist) or a doctor, and assists the training of the trainee by guidance or caregiving. Therefore, the training staff 901 may be called a training instructor, a training caregiver, or a training assistant. The walking training device 100 can also be called a walking training system. The up-down direction, the right-left direction and the front-rear direction in the following description are directions based on the direction of the trainee 900.

The walking training device 100 mainly includes a control panel 133 attached to a frame 130 constituting the entire skeleton, a treadmill 131 on which the trainee 900 walks, and a walking assist device (robot leg) 120 that is attached to an affected leg that is a leg of the trainee 900 on the paralyzed side.

The treadmill 131 is a device that prompts the trainee 900 to walk, and the trainee 900 who performs walking training rides on a belt 1311 and attempts a walking motion in accordance with the movement of the belt 1311. The training staff 901 can stand on the belt 1311 behind the trainee 900 and perform a walking motion together with the trainee 900 as shown in FIG. 1, for example. However, it is usually preferable that the training staff 901 be in a state in which it is easy to perform caregiving to the trainee 900, that is, standing over the belt 1311.

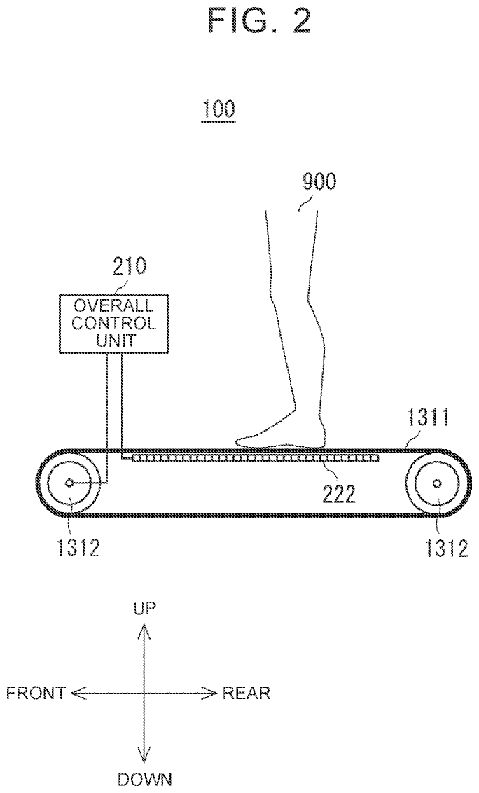

FIG. 2 is a schematic side view of a part of the treadmill 131. As shown in FIG. 2, the treadmill 131 includes at least the ring-shaped belt 1311, a pulley 1312, and a motor (not shown). Further, a load distribution sensor 222 is installed on the inner side of the belt 1311 (on the lower side of the belt 1311 on the surface of which the trainee 900 rides) so as not to move together with the belt 1311.

The load distribution sensor 222 is composed of a plurality of sensors, and these sensors are arranged in a matrix on the lower side of the belt 1311 that supports the sole of the trainee 900. By using these sensors, the load distribution sensor 222 can detect the magnitude and the distribution of the surface pressure (load) received from the sole of the trainee 900. For example, the load distribution sensor 222 is a resistance change detection-type load detection sheet in which a plurality of electrodes is arranged in a matrix. From the detection result of the load distribution sensor 222, it is possible to determine the walking state of the trainee 900 (whether each leg is in a standing state or a swinging state, and the like). The details of the method for estimating the walking state of the trainee 900 based on the detection result of the load distribution sensor 222 will be described later.

In the treadmill 131, for example, an overall control unit 210, which will be described later, determines the walking state of the trainee 900 based on the detection result of the load distribution sensor 222, and uses a motor (not shown) to rotate the pulley 1312 in accordance with the walking state, thereby rotating (moving) the ring-shaped belt 1311. As a result, the trainee 900 can perform walking training without stepping out from the belt 1311.

The frame 130 stands on the treadmill 131 installed on the floor surface, supports the control panel 133 housing the overall control unit 210 that controls the motor and the sensor, and supports a training monitor 138 that is, for example, a liquid crystal panel that presents the training progress and the like to the trainee 900. Further, the frame 130 supports a front tension unit 135 at the front of the overhead portion of the trainee 900, a harness tension unit 112 at the overhead portion, and a rear tension unit 137 at the rear of the overhead portion. The frame 130 also includes handrails 130a for the trainee 900 to grab.

The handrails 130a are arranged on right and left sides of the trainee 900. Each handrail 130a is disposed to extend in a direction parallel to the walking direction of the trainee 900. The position of the handrail 130a in the up-down direction and the right-left direction can be adjusted. That is, the handrails 130a can include a mechanism for changing their height and width. Further, the handrail 130a can be configured such that the height of the handrail 130a is adjusted to make the height of the front side and the height of the rear side in the walking direction different so as to change the inclination angle thereof, for example. For example, the handrail 130a can be provided with an inclination angle that gradually increases along the walking direction.

Further, the handrail 130a is provided with a handrail sensor 218 for detecting the load received from the trainee 900. For example, the handrail sensor 218 can be a resistance change detection-type load detection sheet in which electrodes are arranged in a matrix. Further, the handrail sensor 218 can be a six-axis sensor in which a three-axis acceleration sensor (x,y,z) and a three-axis gyro sensor (roll, pitch, yaw) are combined. However, the type and the installation position of the handrail sensor 218 are not limited.

A camera 140 (imaging device) functions as an imaging unit for observing the whole body of the trainee 900. The camera 140 is installed near the training monitor 138 so as to face the trainee 900. The camera 140 captures still images and moving images of the trainee 900 during training. The camera 140 includes a set of a lens and an imaging element that provides such an angle of view that the whole body of the trainee 900 can be captured. The imaging element is, for example, a complementary metal-oxide-semiconductor (CMOS) image sensor that converts an optical image on an image plane into an image signal.

Here, the camera 140 is installed so as to be able to take an image of at least the surroundings of a region of the belt 1311 of the treadmill 131 on which the trainee 900 rides (in other words, a load detection region of the load distribution sensor 222). This makes it possible to specify whether the load detected by the load distribution sensor 222 is a load received from the sole of the right leg of the trainee 900 or a load received from the sole of the left leg of the trainee 900, from the image taken by the camera 140.

With the coordinated operation of the front tension unit 135 and the rear tension unit 137, the load of the walking assist device 120 is offset so as not to be a burden on the affected leg, and further, the forward swing motion of the affected leg is assisted in accordance with the degree of the setting.

One end of a front wire 134 is connected to a winding mechanism of the front tension unit 135, and the other end is connected to the walking assist device 120. The winding mechanism of the front tension unit 135 winds and unwinds the front wire 134 in accordance with the movement of the affected leg by turning on and off a motor (not shown). Similarly, one end of a rear wire 136 is connected to a winding mechanism of the rear tension unit 137, and the other end is connected to the walking assist device 120. The winding mechanism of the rear tension unit 137 winds and unwinds the rear wire 136 in accordance with the movement of the affected leg by turning on and off a motor (not shown). With such a coordinated operation of the front tension unit 135 and the rear tension unit 137, the load of the walking assist device 120 is offset so as not to be a burden on the affected leg, and further, the forward swing motion of the affected leg is assisted in accordance with the degree of the setting.

For example, as an operator, the training staff 901 sets the level of assistance to high, for a trainee who has severe paralysis. When the assist level is set to high, the front tension unit 135 winds up the front wire 134 with a relatively large force in accordance with the forward swing timing of the affected leg. As the training progresses and assistance becomes no longer needed, the training staff 901 sets the assist level to the minimum. When the assist level is set to the minimum, the front tension unit 135 winds up the front wire 134 with a force to cancel the weight of the walking assist device 120 in accordance with the forward swing timing of the affected leg.

The walking training device 100 further includes a fall prevention harness device composed of a brace 110, a harness wire 111, and a harness tension unit 112.

The brace 110 is a belt wrapped around the abdomen of the trainee 900 and is fixed to the waist portion by, for example, a hook-and-loop fastener. The brace 110 includes a connecting hook 110a for connecting one end of the harness wire 111 that is a hanger, and can also be referred to as a hanger belt. The trainee 900 wears the brace 110 such that the connecting hook 110a is located on the rear back portion.

One end of the harness wire 111 is connected to the connecting hook 110a of the brace 110, and the other end is connected to the winding mechanism of the harness tension unit 112. The winding mechanism of the harness tension unit 112 winds and unwinds the harness wire 111 by turning on and off a motor (not shown). With such a configuration, when the trainee 900 is about to fall, the fall prevention harness device winds up the harness wire 111 in accordance with the instruction of the overall control unit 210 that detects the movement, supports the upper body of the trainee 900 with the brace 110, and suppresses the trainee 900 from falling.

The brace 110 includes a posture sensor 217 for detecting the posture of the trainee 900. The posture sensor 217 is, for example, a combination of a gyro sensor and an acceleration sensor, and outputs an inclination angle of the abdomen on which the brace 110 is attached with respect to the direction of gravity.

The management monitor 139 is a display input device mainly for monitoring and operation by the training staff 901, and is attached to the frame 130. The management monitor 139 is, for example, a liquid crystal panel, and a touch panel is provided on the surface thereof. The management monitor 139 displays various menu items related to training settings, various parameter values at the time of training, training results, and the like. Further, an emergency stop button 232 is provided near the management monitor 139. When the training staff 901 presses the emergency stop button 232, an emergency stop of the walking training device 100 is performed.

The walking assist device 120 is attached to the affected leg of the trainee 900 and assists the trainee 900 in walking by reducing the load of extension and bending at the knee joint of the affected leg. The walking assist device 120 transmits data on the leg movement acquired through walking training to the overall control unit 210, or drives the joint portion in accordance with the instruction from the overall control unit 210. The walking assist device 120 can also be connected to a hip joint (a connecting member including a rotating portion) attached to the brace 110 that is a part of the fall prevention harness device via a wire or the like.

### Details of Walking Assist Device 120

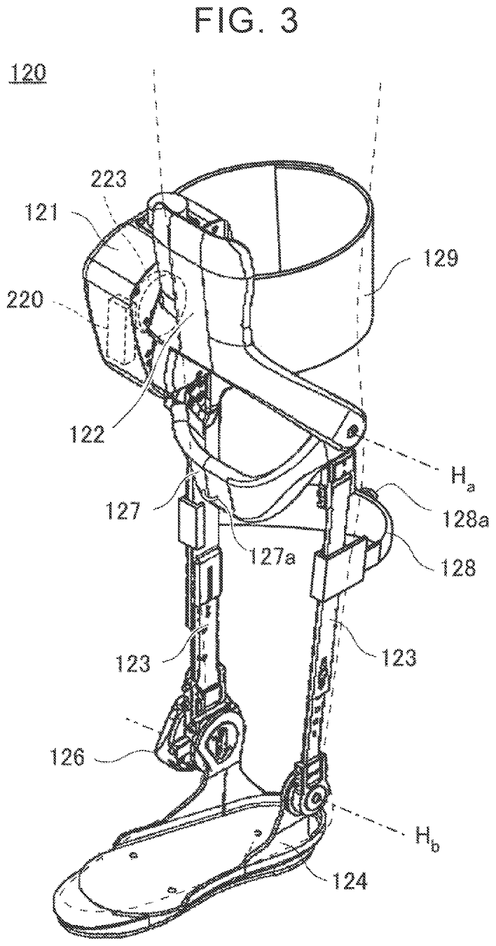

FIG. 3 is a schematic perspective view showing a configuration example of the walking assist device 120. The walking assist device 120 mainly includes a control unit 121 and a plurality of frames that supports various parts of the affected leg. The walking assist device 120 is also referred to as a robot leg.

The control unit 121 includes an auxiliary control unit 220 that controls the walking assist device 120, and also includes a motor (not shown) that generates a driving force for assisting the extension motion and the bending motion of the knee joint. The frames that support various parts of the affected leg include an upper leg frame 122 and lower leg frames 123 that are pivotably connected to the upper leg frame 122. The frames further include a foot flat frame 124 pivotably connected to the lower leg frames 123, a front connecting frame 127 for connecting the front wire 134, and a rear connecting frame 128 for connecting the rear wire 136.

The upper leg frame 122 and the lower leg frames 123 pivot relative to each other around a hinge axis Hₐ shown in the figure. The motor of the control unit 121 rotates following the instruction of the auxiliary control unit 220 to force the upper leg frame 122 and the lower leg frames 123 to relatively open and close around the hinge axis Hₐ. An angle sensor 223 accommodated in the control unit 121 is, for example, a rotary encoder, and detects the angle between the upper leg frame 122 and the lower leg frames 123 around the hinge axis Hₐ. The lower leg frames 123 and the foot flat frame 124 pivot relative to each other around a hinge axis H_{b} shown in the figure. The relative pivot angle range is adjusted in advance by an adjusting mechanism 126.

The front connecting frame 127 is provided so as to extend in the right-left direction on the front side of the upper leg and connect to the upper leg frame 122 at both ends. The front connecting frame 127 is further provided with a connecting hook 127a for connecting the front wire 134, around the center in the right-left direction. The rear connecting frame 128 is provided so as to extend in the right-left direction on the rear side of the lower leg and connect to the lower leg frames 123 at both ends. Further, the rear connecting frame 128 is provided with a connecting hook 128a for connecting the rear wire 136, around the center in the right-left direction.

The upper leg frame 122 is provided with an upper leg belt 129. The upper leg belt 129 is a belt integrally provided on the upper leg frame, and is wrapped around the upper leg portion of the affected leg to fix the upper leg frame 122 to the upper leg portion. This suppresses the entire walking assist device 120 from shifting with respect to the leg of the trainee 900.

### System Configuration Example of Walking Training Device 100

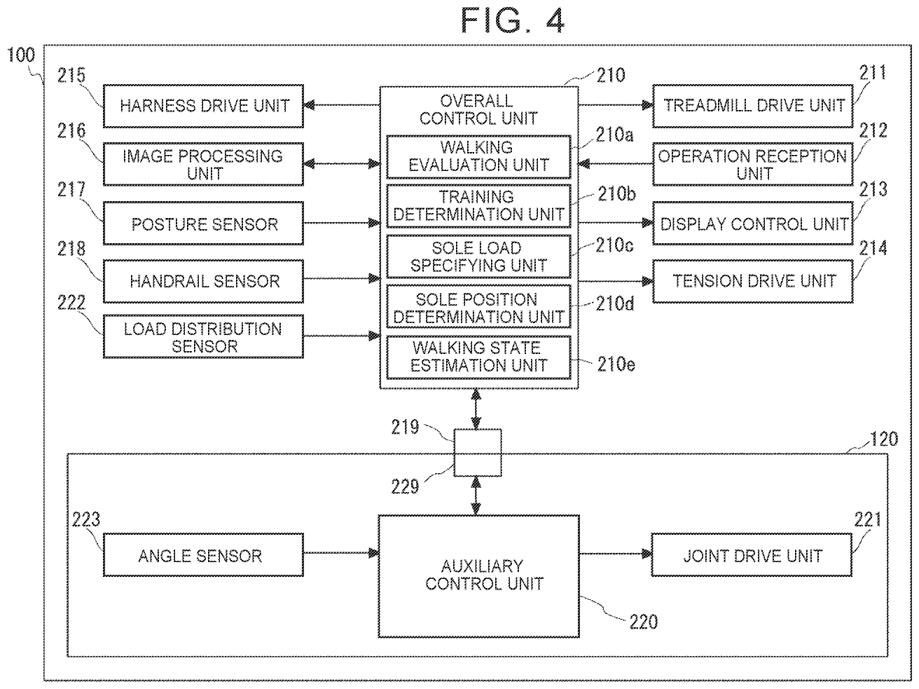

Subsequently, a system configuration example of the walking training device 100 will be described with reference to FIG. 4. FIG. 4 is a block diagram showing the system configuration example of the walking training device 100.

As shown in FIG. 4, the system configuration of the walking training device 100 includes the overall control unit 210, a treadmill drive unit 211, an operation reception unit 212, a display control unit 213, a tension drive unit 214, a harness drive unit 215, an image processing unit 216, the posture sensor 217, the handrail sensor 218, the load distribution sensor 222, a communication connection interface (IF) 219, and the walking assist device 120.

The overall control unit 210 is, for example, a micro processing unit (MPU), and executes control of the entire device by executing a control program read from a system memory.

The treadmill drive unit 211 includes a motor for rotating the belt 1311 of the treadmill 131 and a drive circuit thereof. The overall control unit 210 executes rotation control of the belt 1311 by transmitting a drive signal to the treadmill drive unit 211. The overall control unit 210 adjusts the rotation speed of the belt 1311 in accordance with, for example, the walking speed set by the training staff 901. Alternatively, the overall control unit 210 adjusts the rotation speed of the belt 1311 in accordance with the walking state of the trainee 900 determined based on the detection result of the load distribution sensor 222.

The operation reception unit 212 receives an input operation by the training staff 901 via an operation button provided on the device, a touch panel superimposed on the management monitor 139, an attached remote controller, or the like. The operation signal received by the operation reception unit 212 is transmitted to the overall control unit 210. The overall control unit 210 can give the instruction to switch on and off the power supply or give the instruction to start training based on the operation signal received by the operation reception unit 212. In addition, it is possible to input numerical values related to settings and select menu items. The operation reception unit 212 is not limited to the case where the input operation of the training staff 901 is received, and of course, the operation reception unit 212 can also receive the input operation of the trainee 900.

The display control unit 213 receives a display signal from the overall control unit 210, generates a display image, and displays the image on the training monitor 138 or the management monitor 139. The display control unit 213 generates an image showing the progress of training and a real-time image captured by the camera 140 in accordance with the display signal.

The tension drive unit 214 includes a motor for pulling the front wire 134 and a drive circuit thereof that are provided in the front tension unit 135, and a motor for pulling the rear wire 136 and a drive circuit thereof that are provided in the rear tension unit 137. The overall control unit 210 controls the winding of the front wire 134 and the winding of the rear wire 136 by transmitting a drive signal to the tension drive unit 214. Further, the overall control unit 210 controls the tensile force of each wire by controlling the driving torque of the motor, not limited to the winding operation. Further, the overall control unit 210 identifies the timing at which the affected leg switches from the standing state to the swinging state based on the detection result of the load distribution sensor 222, and increases or decreases the tensile force of each wire in synchronization with that timing, thereby assisting the forward swing motion of the affected leg.

The harness drive unit 215 includes a motor for pulling the harness wire 111 and a drive circuit thereof that are provided in the harness tension unit 112. The overall control unit 210 controls the winding of the harness wire 111 and the tensile force of the harness wire 111 by transmitting a drive signal to the harness drive unit 215. For example, when the trainee 900 is predicted to fall, the overall control unit 210 winds up the harness wire 111 by a certain amount to suppress the trainee from falling.

The image processing unit 216 is connected to the camera 140 and can receive an image signal from the camera 140. The image processing unit 216 receives an image signal from the camera 140 and performs image processing on the received image signal to generate image data, in accordance with the instruction from the overall control unit 210. Further, the image processing unit 216 can also perform image processing on the image signal received from the camera 140 to execute a specific image analysis, in accordance with the instruction from the overall control unit 210. For example, the image processing unit 216 detects the position of the foot (standing position) of the affected leg that is in contact with the treadmill 131 by image analysis. Specifically, for example, the standing position is calculated by extracting an image region near the tip of the foot flat frame 124 and analyzing an identification marker drawn on the belt 1311 that overlaps the tip portion.

As described above, the posture sensor 217 detects the inclination angle of the abdomen of the trainee 900 with respect to the direction of gravity, and transmits the detection signal to the overall control unit 210. The overall control unit 210 calculates the posture of the trainee 900, specifically the inclination angle of the trunk, using the detection signal from the posture sensor 217. The overall control unit 210 and the posture sensor 217 may be connected by wire or by short-range wireless communication.

The handrail sensor 218 detects a load applied to the handrail 130a. That is, a load corresponding to a portion of the weight of the trainee 900 that the trainee 900 cannot support with both legs is applied to the handrail 130a. The handrail sensor 218 detects this load and transmits a detection signal to the overall control unit 210.

As described above, the load distribution sensor 222 detects the magnitude and the distribution of the surface pressure (load) received from the sole of the trainee 900 and transmits the detection signal to the overall control unit 210. The overall control unit 210 receives and analyzes the detection signal to estimate the walking state and estimate switching.

The overall control unit 210 also plays a role as a function execution unit that executes various calculations related to the control and performs the control. The overall control unit 210 includes, for example, a walking evaluation unit 210a, a training determination unit 210b, a sole load specifying unit 210c, a sole position determination unit 210d, and a walking state estimation unit 210e. The sole load specifying unit 210c, the sole position determination unit 210d, and the walking state estimation unit 210e will be described later.

The walking evaluation unit 210a evaluates whether the walking motion of the trainee 900 is abnormal walking using the data acquired from various sensors. The training determination unit 210b determines the training result for a series of walking trainings based on, for example, the cumulative number of abnormal walking evaluated by the walking evaluation unit 210a.

The method of determining the training result and the criteria for determining the training result may be set as appropriate. For example, the training result may be determined by comparing the amount of movement of the paralyzed body portion with the reference for each walking phase. The walking phase is obtained by dividing one walking cycle for the affected leg (or a healthy leg) into a standing phase in which the leg is in the standing state, a transition phase from the standing phase to a swinging phase in which the leg is in the swinging state, a swinging phase, a transition phase from the swinging phase to the standing phase, and so on. The walking phase can be classified (determined) based on, for example, the detection result by the load distribution sensor 222. As described above, for the walking cycle, one cycle can be regarded as including the standing phase, the transition phase, the swinging phase, and the transition phase. However, it does not matter which phase is defined as the start phase. In addition, for the walking cycle, one cycle can be regarded as including, for example, a both leg-supported state, a single leg-(affected leg-)supported state, the both leg-supported state, and a single leg-(healthy leg-)supported state, and in this case, it does not matter which state is defined as the starting state.

In addition, the walking cycle focusing on the right leg or the left leg (healthy leg or affected leg) can be further divided, and can be represented by dividing the standing phase into an initial ground contact and four phases and dividing the swinging phase into three phases. The initial ground contact refers to a moment when an observed foot contacts the floor, and the four phases of the standing phase refer to a load response phase, a standing middle phase, a standing end phase, and a pre-swinging phase. The load response phase is the phase from the initial ground contact to the moment when the foot on the opposite side leaves the floor (contralateral takeoff). The standing middle phase is the phase from the contralateral takeoff to the moment when the heel of the observed foot leaves the floor (heel takeoff). The standing end phase is the phase from the heel takeoff to the initial ground contact on the opposite side. The pre-swinging phase is the phase from the initial ground contact on the opposite side to the time when the observed foot leaves the floor (takeoff). The three phases of the swinging phase refer to a swinging initial phase, a swinging middle phase, and a swinging end phase. The swinging initial phase is the phase from the end of the pre-swinging phase (the above-mentioned takeoff) to the time when both feet cross (feet crossing). The swinging middle phase is the phase from the time when the feet cross to the time when the shinbone becomes vertical (vertical shinbone). The swinging end phase is the phase from the time when the shinbone is vertical to the next initial ground contact.

The communication connection IF 219 is an interface connected to the overall control unit 210, and is an interface for providing a command to the walking assist device 120 attached to the affected leg of the trainee 900 and receiving sensor information.

The walking assist device 120 can include a communication connection IF 229 that is connected to the communication connection IF 219 by wire or wirelessly. The communication connection IF 229 is connected to the auxiliary control unit 220 of the walking assist device 120. The communication connection IF 219 and the communication connection IF 229 are communication interfaces such as a wired local area network (LAN) or a wireless LAN conforming to the communication standards.

Further, the walking assist device 120 can include the auxiliary control unit 220, a joint drive unit 221, and the angle sensor 223. The auxiliary control unit 220 is, for example, an MPU, and controls the walking assist device 120 by executing the control program provided by the overall control unit 210. Further, the auxiliary control unit 220 notifies the overall control unit 210 of the state of the walking assist device 120 via the communication connection IF 219 and the communication connection IF 229. Further, the auxiliary control unit 220 receives a command from the overall control unit 210 and executes control of starting, stopping, and the like of the walking assist device 120.

The joint drive unit 221 includes a motor of the control unit 121 and a drive circuit thereof. The auxiliary control unit 220 transmits the drive signal to the joint drive unit 221 to force the upper leg frame 122 and the lower leg frames 123 to relatively open or close around the hinge axis Hₐ. Such motions assist the knee extension and bending motions and suppress knee collapse.

As described above, the angle sensor 223 detects the angle between the upper leg frame 122 and the lower leg frames 123 around the hinge axis Hₐ, and transmits the detection signal to the auxiliary control unit 220. The auxiliary control unit 220 receives this detection signal and calculates the opening angle of the knee joint.

The walking training device 100 is required to accurately estimate the walking state of the trainee 900 in order to provide effective training to the trainee 900. Here, in order to accurately estimate the walking state of the trainee 900, the reliability of the detection result of the load received from the sole of the trainee 900 needs to be improved. The detection result is referred to when the walking state of the trainee 900 is estimated.

However, in the related art disclosed in, for example, JP 6187208 B, it is not determined whether the user (trainee) is normally walking within the load detection area of the floor reaction force sensor. Therefore, for example, even when the user walks outside the load detection area of the floor reaction force sensor, the sole loads of the right and left lower legs of the user are estimated without considering that fact. Accordingly, in the related art, the reliability of the estimation result of the load received from the sole of the trainee is lowered. As a result, in the related art, there is a possibility that the walking state of the trainee cannot be accurately estimated, for example, and effective walking training cannot be provided to the trainee.

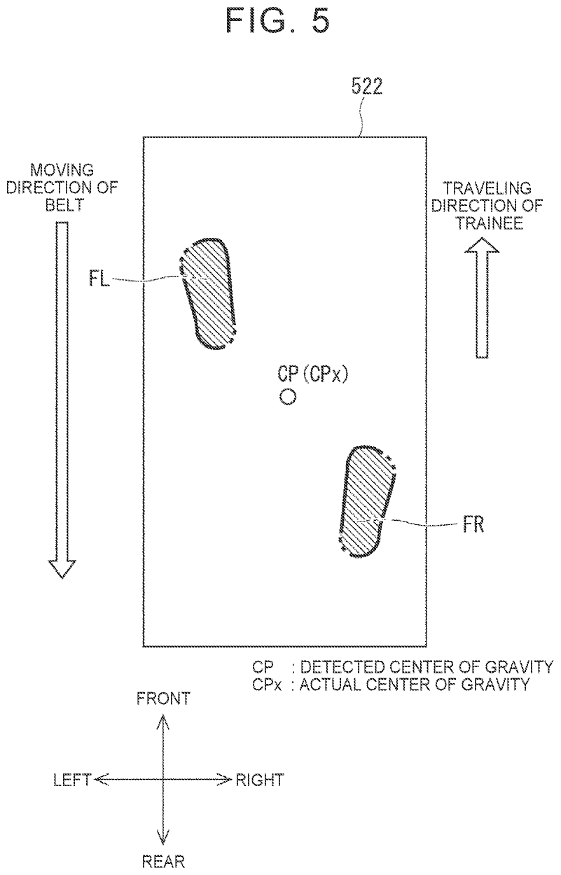

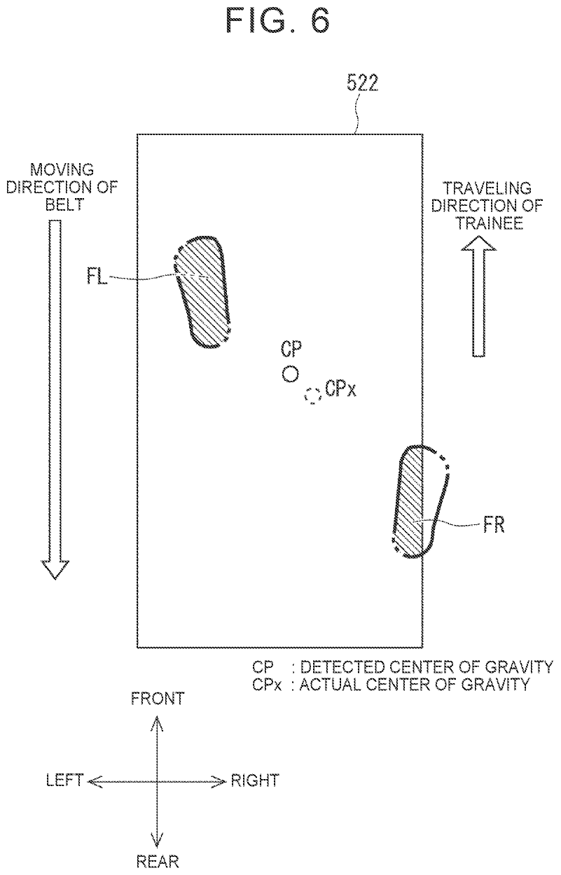

FIGS. 5 and 6 are diagrams illustrating the problem of the method for estimating the walking state of the trainee 900 by the related art. Note that FIG. 5 shows an example of the case where the trainee 900 is normally walking within the load detection area of the floor reaction force sensor 522, and FIG. 6 shows an example of the case where the trainee 900 is walking outside the load detection area of the floor reaction force sensor 522.

As shown in FIG. 5, when the trainee 900 is normally walking within the load detection area of the floor reaction force sensor 522, a center portion between the entire load received from a sole FR of the right leg of the trainee 900 and the entire load received from a sole FL of the left leg of the trainee 900 is detected as the center of gravity CP of the load. The center of gravity CP detected at this time is substantially the same as the center of gravity CPx of the actual load.

In contrast, as shown in FIG. 6, when the trainee 900 is walking with his/her right leg being outside the load detection area of the floor reaction force sensor 522, a center portion between a part of the load received from the sole FR of the right leg of the trainee 900 and the entire load received from the sole FL of the left leg of the trainee 900 is detected as the center of gravity CP of the load. The center of gravity CP detected at this time is closer to the sole FL side of the left leg than the center of gravity CPx of the actual load. In this case, it is determined that the right leg has switched to the swinging state despite the fact that the right leg is still in the standing state. Therefore, if the walking assist device is attached to the right leg, the extension control of the walking assist device may not be performed at an appropriate timing. That is, in the related art, there is a possibility that the walking state of the trainee 900 cannot be accurately estimated and effective walking training cannot be provided to the trainee 900.

Thus, the walking training device 100 according to the present embodiment determines whether the trainee 900 is normally walking within the load detection area of the load distribution sensor 222, and excludes the load received from the sole of the leg of the trainee 900 that is determined as walking outside the load detection area of the load distribution sensor 222, from the reference that is used when estimating the walking state of the trainee 900. That is, the walking training device 100 according to the present embodiment improves the reliability of the detection result of the load received from the sole of the trainee 900. As a result, the walking training device 100 according to the present embodiment can accurately estimate the walking state of the trainee 900, for example, and thus can provide effective walking training to the trainee 900.

Specifically, first, the sole load specifying unit 210c specifies whether the load detected by the load distribution sensor 222 is a load received from the sole of the right leg of the trainee 900 or a load received from the sole of the left leg of the trainee 900, from the image taken by the camera 140. For example, when it is detected from the image taken by the camera 140 that the left leg of the trainee 900 is located forward and the right leg is located rearward, the sole load specifying unit 210c determines that the load of the sole detected at the left front of the load detection area of the load distribution sensor 222 is the load received from the sole of the left leg of the trainee 900, and determines that the load of the sole detected at the right rear is the load received from the sole of the right leg of the trainee 900. After that, based on the status of the load received from the sole of one leg, out of the right leg and the left leg of the trainee 900, in the standing state that is detected by the load distribution sensor 222, the sole position determination unit 210d determines whether the sole of the one leg is located within the load detection area of the load distribution sensor 222.

Then, the walking state estimation unit 210e estimates the walking state of the trainee 900 based on the load received from the sole of each of the right leg and the left leg of the trainee 900. The load is detected by the load distribution sensor 222 and specified by the sole load specifying unit 210c. Here, the walking state estimation unit 210e excludes the load received from the sole of the leg of the trainee 900 that is determined, by the sole position determination unit 210d, as walking outside the load detection area of the load distribution sensor 222, from the reference that is used when estimating the walking state of the trainee 900. Thereby, the walking state estimation unit 210e can accurately estimate the walking state of the trainee 900. As a result, the trainee 900 can perform effective walking training.

### First Example of Method for Determining Sole Position by Sole Position Determination Unit 210d

For example, when the distribution area of the load received from the sole of one leg, out of the right leg and the left leg of the trainee 900 during walking training, in the standing state that is detected by the load distribution sensor 222 is smaller than a predetermined area, the sole position determination unit 210d may determine that the sole of the one leg in the standing state is located outside the load detection area of the load distribution sensor 222. Here, the predetermined area is, for example, an area of the sole detected when the sole of one leg in the standing state is located within the load detection area of the load distribution sensor 222.

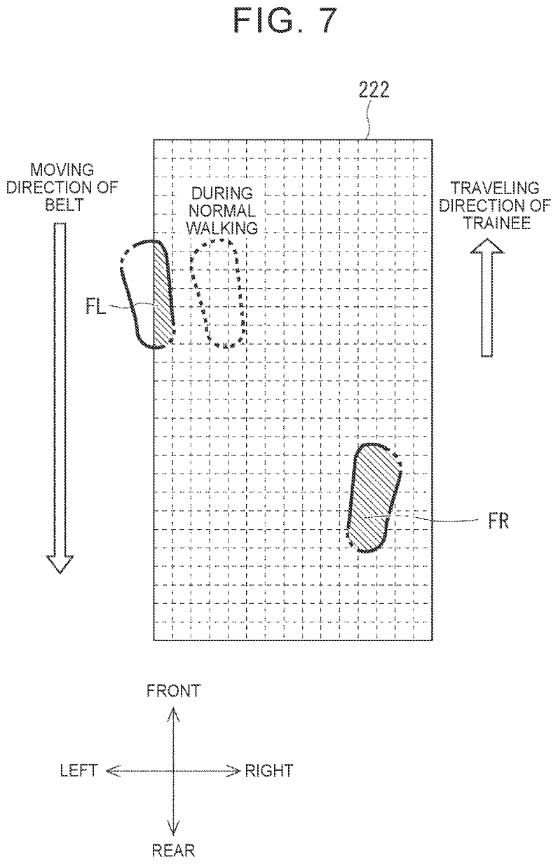

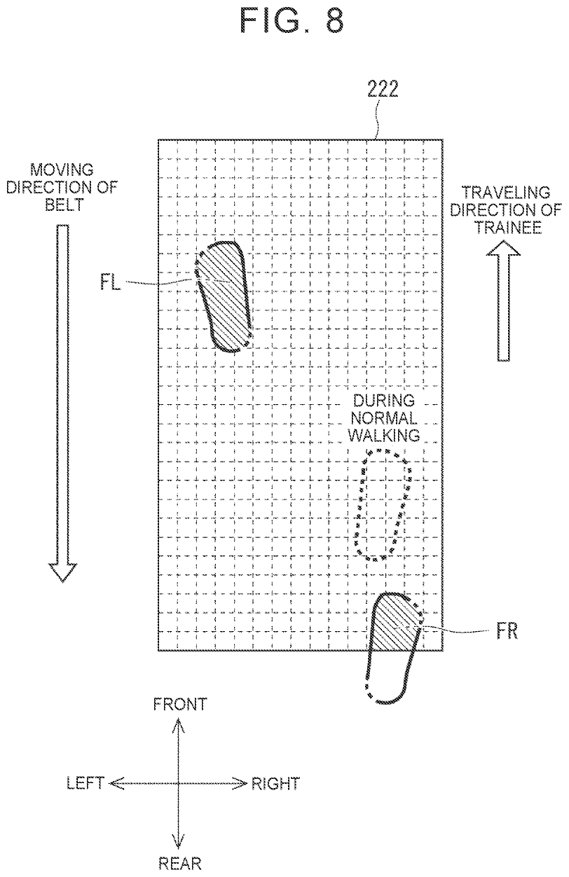

FIGS. 7 and 8 are diagrams illustrating a first example of the method for determining whether the sole of the leg of the trainee 900 in the standing state is located within the load detection area of the load distribution sensor 222 by the sole position determination unit 210d.

In the example of FIG. 7, the distribution area of the load received from the sole FR of the right leg in the standing state is equal to or larger than the predetermined area, so the sole position determination unit 210d determines that the sole FR of the right leg is located within the load detection area of the load distribution sensor 222. In contrast, the distribution area of the load received from the sole FL of the left leg in the standing state is smaller than the predetermined area, so the sole position determination unit 210d determines that the sole FL of the left leg is located outside the load detection area of the load distribution sensor 222.

In the example of FIG. 8, the distribution area of the load received from the sole FL of the left leg in the standing state is equal to or larger than the predetermined area, so the sole position determination unit 210d determines that the sole FL of the left leg is located within the load detection area of the load distribution sensor 222. In contrast, the distribution area of the load received from the sole FR of the right leg in the standing state is smaller than the predetermined area, so the sole position determination unit 210d determines that the sole FR of the right leg is located outside the load detection area of the load distribution sensor 222.

### Second Example of Method for Determining Sole Position by Sole Position Determination Unit 210d

For example, when the load received from the sole of one leg, out of the right leg and the left leg of the trainee 900 during walking training, in the standing state that is detected by the load distribution sensor 222 is smaller than a predetermined load, the sole position determination unit 210d may determine that the sole of the one leg in the standing state is located outside the load detection area of the load distribution sensor 222. Here, the predetermined load is, for example, a load of the sole detected when the sole of one leg in the standing state is located within the load detection area of the load distribution sensor 222.

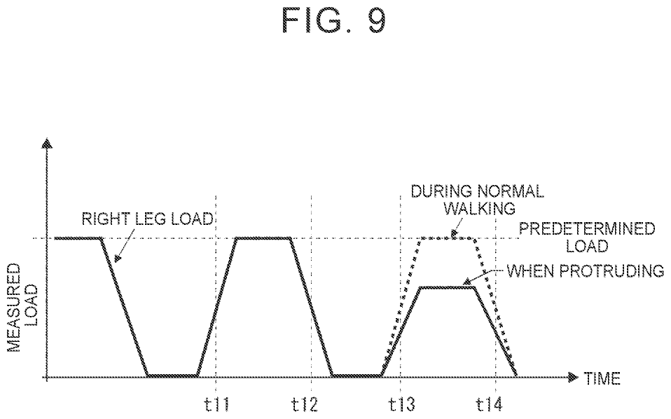

FIG. 9 is a timing chart illustrating a second example of the method for determining whether the sole of the leg of the trainee 900 in the standing state is located within the load detection area of the load distribution sensor 222 by the sole position determination unit 210d. Note that FIG. 9 shows the status of change in the load received from the sole of the right leg of the trainee 900 that is detected by the load distribution sensor 222.

In the example of FIG. 9, when the load received from the sole FR of the right leg of the trainee 900 that is detected by the load distribution sensor 222 is equal to or larger than the predetermined load (time t11 to t12), the sole position determination unit 210d determines that the sole FR of the right leg is located within the load detection area of the load distribution sensor 222. In contrast, when the load received from the sole FR of the right leg of the trainee 900 that is detected by the load distribution sensor 222 is smaller than the predetermined load (time t13 to t14), the sole position determination unit 210d determines that the sole FR of the right leg is located outside the load detection area of the load distribution sensor 222.

### Third Example of Method for Determining Sole Position by Sole Position Determination Unit 210d

For example, when the load received from the sole of one leg, out of the right leg and the left leg of the trainee 900 during walking training, in the standing state is detected in an end area that is set along an outer periphery of the load detection area of the load distribution sensor 222, the sole position determination unit 210d may determine that the sole of the one leg in the standing state is located outside the load detection area of the load distribution sensor 222.

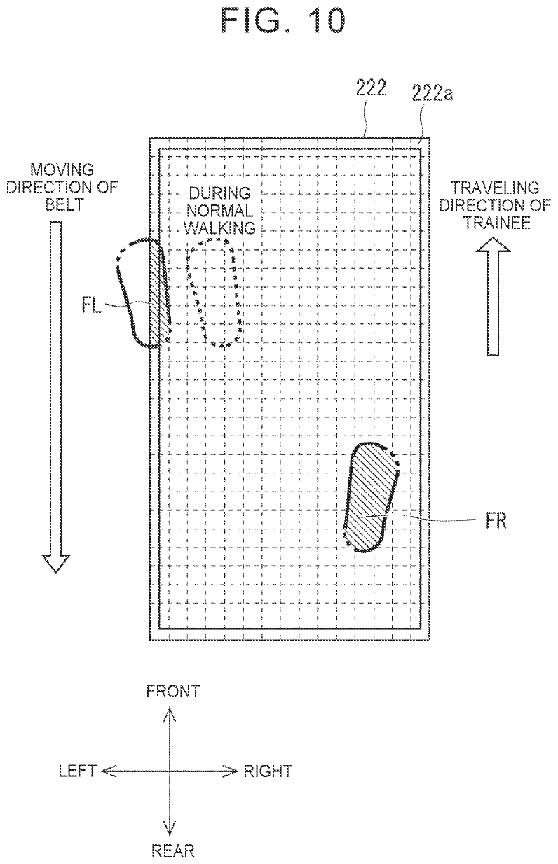

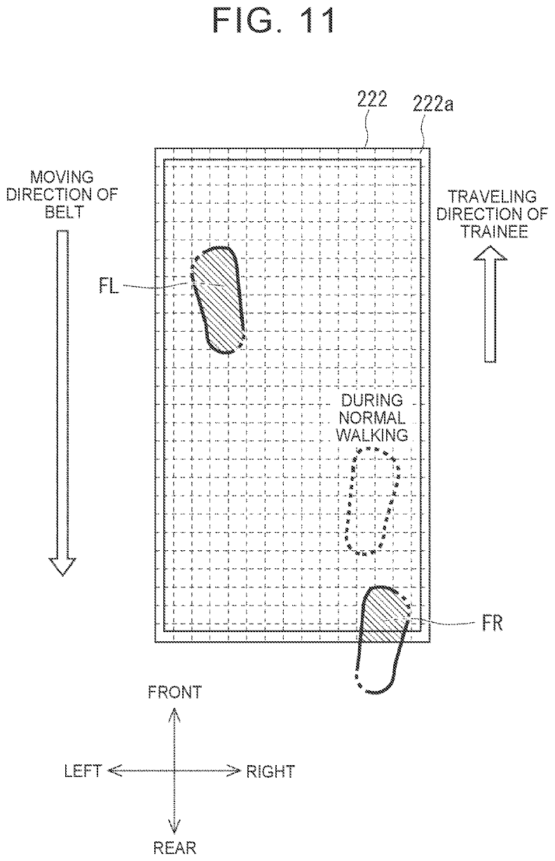

FIGS. 10 and 11 are diagrams illustrating a third example of the method for determining whether the sole of the leg of the trainee 900 in the standing state is located within the load detection area of the load distribution sensor 222 by the sole position determination unit 210d.

In the example of FIG. 10, the load received from the sole FR of the right leg in the standing state is not detected in an end area 222a set along the outer periphery of the load detection area of the load distribution sensor 222, so the sole position determination unit 210d determines that the sole FR of the right leg is located within the load detection area of the load distribution sensor 222. In contrast, the load received from the sole FL of the left leg in the standing state is detected in the end area 222a, so the sole position determination unit 210d determines that the sole FL of the left leg is located outside the load detection area of the load distribution sensor 222.

In the example of FIG. 11, the load received from the sole FL of the left leg in the standing state is not detected in the end area 222a set along the outer periphery of the load detection area of the load distribution sensor 222, so the sole position determination unit 210d determines that the sole FL of the left leg is located within the load detection area of the load distribution sensor 222. In contrast, the load received from the sole FR of the right leg in the standing state is detected in the end area 222a, so the sole position determination unit 210d determines that the sole FR of the right leg is located outside the load detection area of the load distribution sensor 222.

Subsequently, a method for estimating the walking state of the trainee 900 by the walking state estimation unit 210e when the sole position determination unit 210d determines that the trainee 900 is walking outside the load detection area of the load distribution sensor 222 will be described.

### First Example of Method for Estimating Walking State by Walking State Estimation Unit 210e

For example, when the sole position determination unit 210d determines that the sole of one leg is located outside the load detection area of the load distribution sensor 222, the walking state estimation unit 210e may estimate the walking state of the one leg based on the change in the load received from the sole of the other leg that is detected by the load distribution sensor 222.

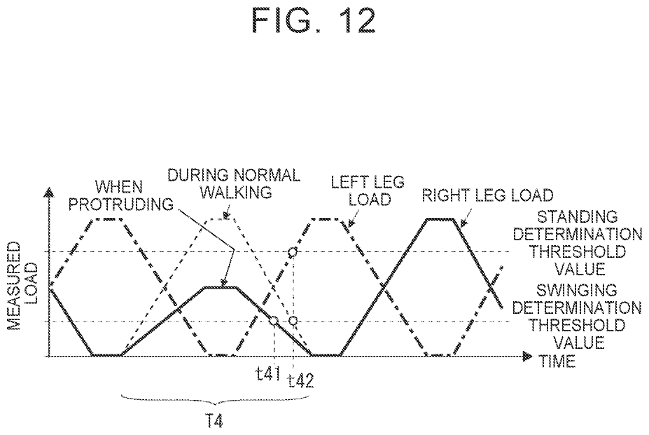

FIG. 12 is a timing chart illustrating a first example of the method for estimating the walking state of the trainee 900 by the walking state estimation unit 210e.

In the example of FIG. 12, in a period T4, the sole of the right leg is located outside (protruding from) the load detection area of the load distribution sensor 222. Here, if the walking state of the right leg is estimated based on the change in the load received from the sole of the right leg, the switching time (timing(time t41)) of the right leg from the standing state to the swinging state is estimated to be earlier than the switching timing during normal walking (time t42). Thus, in the present embodiment, the walking state of the right leg protruding from the load detection area is estimated based on the change in the load received from the sole of the left leg rather than the right leg.

Specifically, first, the load value of the left leg at the timing of switching of the right leg from the standing state to the swinging state during normal walking (when walking within the load detection area) is acquired in advance. Then, the walking state estimation unit 210e estimates the time (timing) when the load of the left leg reaches a predetermined load acquired in advance as the time when the right leg switches from the standing state to the swinging state (time t43). Thereby, the walking state estimation unit 210e can accurately estimate the walking state of the trainee 900.

### Second Example of Method for Estimating Walking State by Walking State Estimation Unit 210e

For example, when the sole position determination unit 210d determines that the sole of one leg is located outside the load detection area of the load distribution sensor 222, the walking state estimation unit 210e may estimate the walking state of the one leg based on information on the past load change of the one leg.

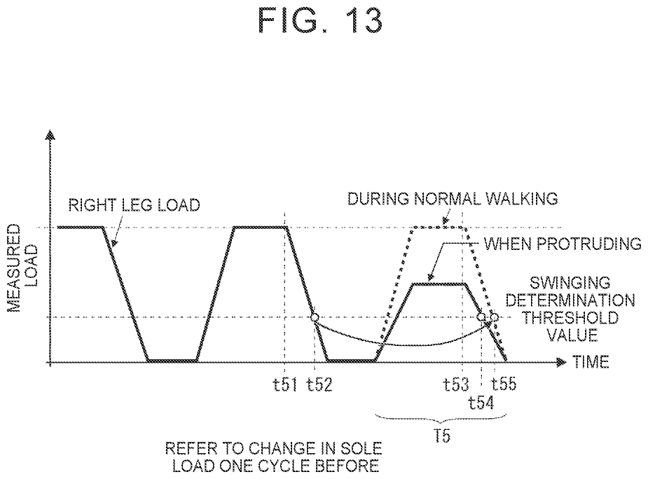

FIG. 13 is a timing chart illustrating a second example of the method for estimating the walking state of the trainee 900 by the walking state estimation unit 210e.

In the example of FIG. 13, in a period T5, the sole of the right leg is located outside (protruding from) the load detection area of the load distribution sensor 222. Here, if the walking state of the right leg is estimated based on the change in the load received from the sole of the right leg, the switching time (timing(time t54)) of the right leg from the standing state to the swinging state is estimated to be earlier than the switching timing during normal walking (time t55). Thus, in the present embodiment, the walking state of the right leg protruding from the load detection area is estimated based on the change in the sole load of the right leg one cycle before.

Specifically, the walking state estimation unit 210e adopts the time (time t51 to t52) from the time when the sole load of the right leg one cycle before starts decreasing to the time when the sole load reaches a swinging determination threshold value as the time (time t53 to t55) from the time when the sole load of the right leg protruding from the load detection area starts decreasing to the time when the right leg has switched to the swinging state. Thereby, the walking state estimation unit 210e can accurately estimate the walking state of the trainee 900.

Third Example of Method for Estimating Walking State by Walking State Estimation Unit 210e

For example, when the sole position determination unit 210d determines that the sole of one leg is located outside the load detection area of the load distribution sensor 222, the walking state estimation unit 210e may estimate the walking state of the one leg based on information on the change in the average load of the one leg during normal walking.

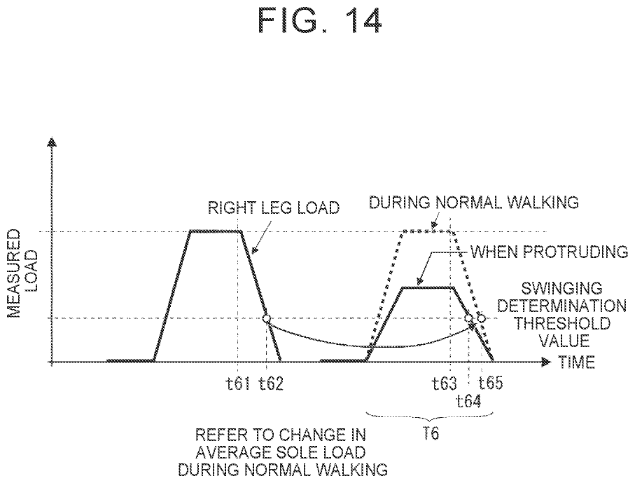

FIG. 14 is a timing chart illustrating a third example of the method for estimating the walking state of the trainee 900 by the walking state estimation unit 210e.

In the example of FIG. 14, in a period T6, the sole of the right leg is located outside (protruding from) the load detection area of the load distribution sensor 222. Here, if the walking state of the right leg is estimated based on the change in the load received from the sole of the right leg, the switching time (timing(time t64)) of the right leg from the standing state to the swinging state is estimated to be earlier than the switching timing during normal walking (time t65). Thus, in the present embodiment, the walking state of the right leg protruding from the load detection area is estimated based on the change in the average load of the right leg during normal walking.

Specifically, the time (time t61 to t62) from the time when the average sole load of the right leg during normal walking starts decreasing to the time when the average sole load reaches a swinging determination threshold value is adopted as the time (time t63 to t65) from the time when the sole load of the right leg protruding from the load detection area starts decreasing to the time when the right leg has switched to the swinging state. Thereby, the walking state estimation unit 210e can accurately estimate the walking state of the trainee 900.

As described above, the walking training device 100 according to the present embodiment determines whether the trainee 900 is normally walking within the load detection area of the load distribution sensor 222, and excludes the load received from the sole of the leg of the trainee 900 that is determined as walking outside the load detection area of the load distribution sensor 222, from the reference that is used when estimating the walking state of the trainee 900. That is, the walking training device 100 according to the present embodiment improves the reliability of the detection result of the load received from the sole of the trainee 900. As a result, the walking training device 100 according to the present embodiment can accurately estimate the walking state of the trainee 900, for example, and thus can provide effective walking training to the trainee 900.

Further, in each of the above embodiments, the case where the trainee 900 is a hemiplegic patient suffering from paralysis in one leg has been described as an example, but the present disclosure is not limited to this. The trainee 900 may be, for example, a patient suffering from paralysis of both legs. In that case, the trainee 900 performs training while wearing the walking assist device 120 on both legs. Alternatively, the trainee 900 does not have to wear the walking assist device 120 on any of the legs.

Further, in the present disclosure, part or all of the processes in the walking training device 100 can be realized by causing a central processing unit (CPU) to execute a computer program.

The above program includes instructions (or software codes) for causing the computer to perform one or more of the functions described in the embodiments when loaded into the computer. The program may be stored in a non-transitory computer-readable medium or a tangible storage medium. Examples of the non-transitory computer-readable medium or the tangible storage medium include, but are not limited to, a random-access memory (RAM), a read-only memory (ROM), a flash memory, a solid-stated drive (SSD) or other memory technologies, a compact disc read-only memory (CD-ROM), a digital versatile disc (DVD), Blu-ray (registered trademark) disc, or other optical disc storages, and a magnetic cassette, a magnetic tape, a magnetic disc storage or other magnetic storage devices.

The program may be transmitted on a transitory computer-readable medium or a communication medium. Examples of the transitory computer-readable medium or the communication medium include, but are not limited to, electrical, optical, acoustic, or other forms of propagating signals.

What is claimed is:

1. A walking training system comprising:
a treadmill;
a load distribution sensor that is provided on a lower side of a belt of the treadmill so as not to move together with the belt and that detects a distribution of a load received from a sole of a trainee riding on the belt of the treadmill;
an imaging device that takes an image of the trainee;
a specifying unit that specifies, from the image taken by the imaging device, whether the load detected by the load distribution sensor is a load received from a sole of a right leg of the trainee or a load received from a sole of a left leg of the trainee; and
a determination unit that determines, based on a status of a load received from a sole of one leg, out of the right leg and the left leg of the trainee during walking training, in a standing state that is detected by the load distribution sensor, whether the sole of the one leg in the standing state is located within a load detection area of the load distribution sensor.

2. The walking training system according to claim 1, wherein when the load received from the sole of the one leg, out of the right leg and the left leg of the trainee during walking training, in the standing state that is detected by the load distribution sensor is smaller than a predetermined load, the determination unit determines that the sole of the one leg in the standing state is located outside the load detection area of the load distribution sensor.

3. The walking training system according to claim 1, wherein when a distribution area of the load received from the sole of the one leg, out of the right leg and the left leg of the trainee during walking training, in the standing state that is detected by the load distribution sensor is smaller than a predetermined area, the determination unit determines that the sole of the one leg in the standing state is located outside the load detection area of the load distribution sensor.

4. The walking training system according to claim 1, wherein when the load received from the sole of the one leg, out of the right leg and the left leg of the trainee during walking training, in the standing state that is detected by the load distribution sensor is detected in an end area that is set along an outer periphery of the load detection area of the load distribution sensor, the determination unit determines that the sole of the one leg in the standing state is located outside the load detection area of the load distribution sensor.

5. The walking training system according to claim 1, further comprising an estimation unit that estimates a walking state of the trainee based on a load received from a sole of each of the right leg and the left leg of the trainee that is detected by the load distribution sensor and specified by the specifying unit.

6. The walking training system according to claim 5, wherein when the determination unit determines that the sole of the one leg is located outside the load detection area of the load distribution sensor, the estimation unit estimates a walking state of the one leg based on a change in a load received from a sole of another leg that is detected by the load distribution sensor.

7. The walking training system according to claim 5, wherein when the determination unit determines that the sole of the one leg is located outside the load detection area of the load distribution sensor, the estimation unit estimates a walking state of the one leg based on information on a past load change of the one leg.

8. The walking training system according to claim 5, further comprising:
a robot leg attached to at least one leg of the trainee; and
a control unit that controls extension of the robot leg based on an estimation result by the estimation unit.

9. A method for controlling a walking training system, the method comprising:
a step of using a load distribution sensor that is provided on a lower side of a belt of a treadmill so as not to move together with the belt, to detect a distribution of a load received from a sole of a trainee riding on the belt of the treadmill;
a step of taking an image of the trainee using an imaging device;
a step of specifying, from the image taken by the imaging device, whether the load detected by the load distribution sensor is a load received from a sole of a right leg of the trainee or a load received from a sole of a left leg of the trainee; and
a step of determining, based on a status of a load received from a sole of one leg, out of the right leg and the left leg of the trainee during walking training, in a standing state that is detected by the load distribution sensor, whether the sole of the one leg in the standing state is located within a load detection area of the load distribution sensor.

10. A control program that causes a computer to execute:
a process of using a load distribution sensor that is provided on a lower side of a belt of a treadmill so as not to move together with the belt, to detect a distribution of a load received from a sole of a trainee riding on the belt of the treadmill;
a process of taking an image of the trainee using an imaging device;
a process of specifying, from the image taken by the imaging device, whether the load detected by the load distribution sensor is a load received from a sole of a right leg of the trainee or a load received from a sole of a left leg of the trainee; and
a process of determining, based on a status of a load received from a sole of one leg, out of the right leg and the left leg of the trainee during walking training, in a standing state that is detected by the load distribution sensor, whether the sole of the one leg in the standing state is located within a load detection area of the load distribution sensor.
